(19) United States
(12) Patent Application Publication
(10) Pub. No.: US 2017/0209171 A1
(43) Pub. Date: Jul. 27, 2017

(54) DETACHABLE ANCHOR FOR GUIDING TOOL OF MINIMALLY INVASIVE SURGERY

(71) Applicant: Medishield B.V., Delft (NL)

(72) Inventors: Tim Horeman, Leiden (NL); Willem Nerkens, Den Haag (NL)

(73) Assignee: Medishield B.V., Delft (NL)

(21) Appl. No.: 15/506,958

(22) PCT Filed: Aug. 27, 2015
(86) PCT No.: PCT/EP2015/025061

§ 371 Date: Feb. 27, 2017

(30) Foreign Application Priority Data
Aug. 27, 2014 (GB) 1415188.0

(51) Int. Cl. A61B 17/34

(52) U.S. Cl. CPC A61B 17/3417; A61B 17/34; A61B 2017/3484; A61B 2017/3419; A61B 2017/3435; A61B 2017/348; A61B 2017/3486

(57) ABSTRACT

Disclosed is a detachable anchor for a guiding tool of a minimally invasive surgery. The detachable anchor includes a hollow elongate member arranged to coaxially fit over a shaft of the guiding tool. The detachable anchor also includes an expandable sleeve having a first end and a second end. The expandable sleeve is at least partially double wall structure consisting of an inner wall and an outer wall. The walls are attached to each other at least at the second end of the sleeve. Further, the sleeve is attached at its first end to the hollow clongate member and has an inlet for inflating medium.

### TECHNICAL FIELD

The present disclosure generally relates to the field of surgical devices, and more specifically to a detachable anchor for a guiding tool used in a minimally invasive surgery and a method for anchoring the guiding tool to an incision using the detachable anchor.

### BACKGROUND

Minimally invasive or laparoscopic surgical procedure has become the first choice for a wide range of surgical procedures because of several advantages, such as reduced postoperative pain, improved cosmetic results and reduced hospital stays. Generally, such laparoscopic surgery utilizes small incisions and surgical tools, such as graspers, scissors and staplers, configured to be inserted through these incisions.

Typically, guiding tools, such as trocars or cannulas, are inserted through these incisions for providing access path for the surgical tools to reach the target tissues. Often, such incisions are made large enough such that the guiding tools can pass there-through.

However, during the surgical procedure the manipulation of the surgical tools around the incisions and/or the use of large surgical tools can enlarge or widen such incisions. This can reduce fixation or anchoring of the guiding tools around these incisions. In such instance, controlling an insertion depth for the guiding tools becomes difficult as moving the guiding tools can further widen the incisions and the loosened guiding tool can slip out. Additionally, widening or loosening of the incisions can cause leakage of insufflation gas from around the guiding tools. Such conditions are not favourable and can increase workload on the surgeons and can make the surgical procedure challenging.

Therefore, in light of the foregoing discussion, there exists a need to overcome the aforementioned drawbacks of anchoring a guiding tool to an incision of a minimally invasive surgery. Further, it is desirable to have a cost effective solution for such anchoring of the guiding tool to the incision.

### BRIEF SUMMARY

The present disclosure seeks to at least partially overcome the aforementioned drawbacks by providing a detachable anchor for a guiding tool of a minimally invasive surgery.

In one aspect, the present disclosure provides a detachable anchor for a guiding tool of a minimally invasive surgery. The detachable anchor typically comprises a hollow elongate member arranged to coaxially fit over a shaft of the guiding tool. The detachable anchor also comprises an expandable sleeve having a first end and a second end. The expandable sleeve has at least partially double wall structure consisting of an inner wall and an outer wall. The walls are attached to each other at least at the second end of the sleeve. Further, the sleeve is attached at its first end to the hollow elongate member and has an inlet for inflating medium.

In another aspect, the present disclosure provides a method for arranging a detachable anchor over a guiding tool of a minimally invasive surgery. The method typically comprises steps of arranging a detachable anchor, having a hollow elongate member and an expandable sleeve, over a shaft of a guiding tool with the expandable sleeve rolled up next to the hollow elongate member and thereafter, unrolling the expandable sleeve over the shaft of the guiding tool.

Additional aspects, advantages, features and objects of the present disclosure will be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

FIG. 1 is an isometric view of a detachable anchor for a guiding tool of a minimally invasive surgery, in accordance with an embodiment of the present disclosure;

FIG. 2 is an isometric view of the detachable anchor with an expandable sleeve thereof in an unrolled state, in accordance with an embodiment of the present disclosure;

FIGS. 3A-3B are cross-sectional views of a hollow elongate member of the detachable anchor with a first end of the expandable sleeve attached to the hollow elongate member, in accordance with an embodiment of the present disclosure;

FIG. 4 is an isometric view of the detachable anchor arranged on a shaft of the guiding tool, such as a trocar, with the expandable sleeve in a rolled state, in accordance with an embodiment of the present disclosure

FIG. 5 is an isometric view of the detachable anchor arranged on the shaft of the guiding tool with the expandable sleeve in the unrolled state, in accordance with an embodiment of the present disclosure;

FIG. 6 is an isometric view of the detachable anchor arranged on the shaft of the guiding tool with the expandable sleeve in an inflated state, in accordance with an embodiment of the present disclosure;

FIG. 7 is a sectional view of a female abdominal cavity with detachable anchors anchoring guiding tools in surgical incisions of a laparoscopic surgical procedure, in accordance with an embodiment of the present disclosure; and

FIGS. 8A-E show some steps of a method for anchoring a guiding tool, using a detachable anchor of the present disclosure, to a surgical incision of a minimally invasive surgery; in accordance with an embodiment of the present disclosure.

### DETAILED DESCRIPTION OF EMBODIMENTS OF THE DISCLOSURE

The following detailed description illustrates embodiments of the present disclosure and ways in which they can be implemented. Although the best mode of carrying out the present disclosure has been disclosed, those in the art would recognize that other embodiments for carrying out or practicing the present disclosure are also possible.

In one aspect, embodiments of the present disclosure disclose a detachable anchor for a guiding tool for a minimally invasive surgery. The term “guiding tool” used herein refers to a tool which is used in the minimally invasive surgery for primarily providing an access path for a surgical tool (for example graspers, scissors and staplers) to reach the target tissue. The guiding tool is configured to be inserted through a surgical incision, made during the minimally invasive surgery, for defining the access path through which the surgical tool can be inserted. In an example, the guiding tool can be an elongated hollow tool, which can be inserted through the surgical incision, and configured to receive the surgical tool there-through. For example, the guiding tool can be a trocar or a cannula. Alternatively, the guiding tool can be an arthroscope used in an arthroscopic surgical procedure or a catheter to be inserted through an abdominal wall.

The detachable anchor typically comprises a hollow elongate member arranged to coaxially fit over a shaft of the guiding tool. The detachable anchor also typically comprises an expandable sleeve having a first end and a second end as well as having at least partially double wall structure consisting of an inner wall and an outer wall, wherein the walls are attached to each other at least at the second end of the sleeve. The sleeve is further attached at its first end to the hollow elongate member, and has an inlet for inflating medium.

In an embodiment, the second end of the expandable sleeve may have a single wall, for example, at the second end of the expandable sleeve the inner wall and the outer wall may be fused together to form the single wall.

According to an embodiment, the hollow elongate member includes a circular cross-section, i.e. the hollow elongate member is a tubular structure. Alternatively, the hollow elongate member may have oval or polygonal cross-section.

In an embodiment, the hollow elongate member comprises a first portion, a second portion integrally extending from the first portion and an opening extending through the first portion and the second portion, wherein the first portion and second portion are co-axial. The first portion optionally includes a thickness larger as compared to a thickness of the second portion, and a length smaller as compared to a length of the second portion. The opening preferably has a uniform diameter throughout the first portion and the second portion. The hollow elongate member may be made of a substantially hard biocompatible material selected from the group consisting of metal, plastic, glass, ceramic, fiber-reinforced plastic or any combination thereof.

The detachable anchor also typically comprises an expandable sleeve having a first end and a second end. The first end of the expandable sleeve is defined to be a proximal end with respect to the hollow elongate member. The second end of the expandable sleeve is thus defined to be a distal end with respect to the hollow elongate member, for example, when the detachable anchor is arranged over the guiding tool and the expandable sleeve is unrolled over a shaft of the guiding tool.

The expandable sleeve is preferably tubular in shape. Further, the expandable sleeve has a double wall structure consisting of an inner wall and an outer wall. The walls are attached to each other at least at the second end of the sleeve. Also, the expandable sleeve is attached at its first end to the hollow elongate member and has an inlet for inflating medium at the first end.

In one embodiment, a band is arranged to secure the first end of the expandable sleeve to the hollow elongate member. The band secures the first end of the expandable sleeve over the second portion, adjacent to the first portion. Further, the expandable sleeve is most preferably elastic in nature, i.e. made of an elastic material. For example, the expandable sleeve can be made of an elastic biocompatible material selected from the group consisting of elastic polymeric material, such as silicone, polyisoprene, polyurethane, rubber, latex and elastic composite polymeric material.

The tubular structure and the preferred elastic nature of the expandable sleeve allow the expandable sleeve to snugly fit over the hollow elongate member. Specifically, a diameter of the expandable sleeve can be smaller as compared to a diameter of the second portion of the hollow elongate member; therefore the expandable sleeve can snugly fit over the second portion. Moreover, in one embodiment, the inner wall of the expandable sleeve at its first end can be attached to an outer surface of the second portion using an adhesive for further strengthening the attachment between the expandable sleeve and the hollow elongate member.

In one embodiment, the diameter of the expandable sleeve, preferably in the rolled up state, can be larger as compared to the diameter of the second portion of the hollow elongate member. Specifically, the expandable sleeve can be rolled on an external application element, such as a ring, having larger diameter as compared to the diameter of the second portion of the hollow elongate member. Accordingly, the expandable sleeve (in the rolled up state) can be easily arranged on the second portion of the hollow elongate member. Further, the external application element can be removed when the expandable sleeve is unrolled over the shaft of the guiding tool.

Generally, the sleeve can be arranged to be rolled up towards its first end next to the hollow elongate member, when the detachable anchor is in an unutilized state. For example, the expandable sleeve is rolled up to fit over the second portion next to the first portion in the unutilized state thereof.

According to an embodiment, the hollow elongate member further comprises an inlet and an outlet for inflating medium. The outlet is connected to the inlet for inflating medium in the expandable sleeve. The outlet of the hollow elongate member and the inlet of the expandable sleeve are preferably configured to have an airtight fluidic coupling there-between such that when an inflating medium is delivered there-through there is no leakage of the inflating medium from there-around. For example, the first end of the expandable sleeve can be such that both walls are attached to each other and then the expandable sleeve is attached to the hollow elongate member, with an opening for the inlet of the inflating medium. Alternatively, both walls can be attached separately to the hollow elongate member so that the inflating medium can enter in between the walls through an entire circumference of the expandable sleeve about the first end.

The detachable anchor according to an embodiment of the present disclosure comprises a connector or valve adapted to be coupled to the inlet of the hollow elongate member. In one embodiment, the connector can be an integral portion of the hollow elongate member instead of being a separate component. The connector is adapted to connect the hollow elongate member of the detachable anchor to an inflating arrangement for inflating the expandable sleeve of the detachable anchor. In an example, the inflating arrangement includes an elongated pipe adapted to be coupled to the connector and a syringe adapted to be connected to the elongated pipe and carrying the inflating medium therein.

In one embodiment, the thickness of at least one of the two walls of the expandable sleeve varies along its length. The length of the sleeve is a distance between its first and second ends. The thickness of at least one of the walls of the expandable sleeve may be smaller at its second end than at its first end. Moreover, the thickness of only one wall can vary along its length. For example, the outer wall can have varying thickness (smaller thickness at its second end than at its first end) along its length, whereas the inner wall can have a uniform thickness along the length thereof.

The expandable sleeve can be inflated with the help the inflating arrangement using the inflating medium. The inflating medium can be a liquid or gas. In one embodiment, the inflating medium can be air which can be delivered to the expandable sleeve with the help of the inflating arrangement, i.e. the elongated pipe and the syringe. Alternatively, water, saline solution or an inert gas can be used as the inflating medium.

The expandable sleeve preferably inflates non-uniformly due to varying thickness of the walls thereof. As mentioned herein, the thickness of the inner wall of the expandable sleeve can be smaller at the second end as compared to the thickness of the inner wall at the first end. Therefore, the second end of the expandable sleeve can inflate more compared to the first end of the expandable sleeve, when the inflating medium is delivered into the expandable sleeve by the inflating arrangement. Moreover, the non-uniform expansion of the sleeve may be accomplished with the use of polymers with different elastic properties. For example, the expandable sleeve may be made of composite polymers having varying elastic properties at different locations based on its molecular structure.

According to yet another embodiment, the sleeve is arranged to be rolled up towards its first end. This makes it easier to combine the anchor with a guiding tool, and the sleeve can be rolled up while inserting the anchor over a shaft of the guiding tool, and further rolled down once the hollow elongate member is correctly positioned over the shaft.

The detachable anchor according to an embodiment of the present disclosure comprises a spacer arranged between the two walls of the expandable sleeve. The spacer can be a wire, a tube or a thick protrusion from the expandable sleeve. The spacer extends from its first end towards its second end. According to an embodiment, the spacer extends on at least 80% of the length of the expandable sleeve. The spacer helps the inflating medium to suitably flow along the length of the expandable sleeve. Specifically, the spacer is configured to distribute the inflating medium evenly along the length of the expandable sleeve. For example, the spacer provides small openings on either side thereof, and the openings help the inflating medium to suitably flow along the length of the expandable sleeve when the inflating medium is delivered to the expandable sleeve by the inflating arrangement. The spacer is preferably flexible in nature, since the spacer is adapted to be placed between the walls of the expandable sleeve and the expandable sleeve is configured to be rolled in the unutilized state. The spacer can be made of a flexible biocompatible material selected from the group consisting of flexible polymeric material, such as silicone rubber, polytetrafluoroethylene, polyethylene, polypropylene or fixable composite polymeric material.

In one embodiment, the detachable anchor optionally includes a tube, instead of or in addition to the spacer, arranged between the two walls of the expandable sleeve. The tube extends from its first end towards its second end and is connected to the inlet of inflating medium. The tube is thus arranged to conduct the inflating medium into the expandable sleeve. The tube may include a plurality of holes arranged along its length. The plurality of holes is thus configured in deliver the inflating medium into the expandable sleeve when the detachable anchor is fluidically connected to the inflating arrangement.

The present description also relates to a method for arranging a detachable anchor according to this description, over a guiding tool of a minimally invasive surgery, comprising the steps of arranging the detachable anchor over a shaft of the guiding tool, while the sleeve of the detachable anchor is rolled up next to the hollow elongate member, and unrolling the sleeve over the shaft of the guiding tool.

The detachable anchor can thus be easily arranged on the guiding tool, such as a trocar. Initially, the guiding tool is inserted through the detachable anchor while the sleeve is rolled up, and thereafter the expandable sleeve of the detachable anchor is unrolled over the shaft of the guiding tool for snugly securing the detachable anchor to the guiding tool. The guiding tool along with the detachable anchor can be inserted through an incision of a laparoscopic surgical procedure. The detachable anchor accordingly hermetically anchors the guiding tool to the incision when the expandable sleeve is inflated with the inflating medium. Therefore, the guiding tool is configured to have a very limited movement with respect to the surgical incision even when an upward or a downward force is applied on the guiding tool. The detachable anchor accordingly avoids slipping or any undesirable movement of the guiding tool with respect to the incision. Moreover, the detachable anchor hermetically seals the surgical incision to avoid any leakage of insufflation gas from within a patient's body through the surgical incision.

The trocar can be easily maneuvered about the surgical incision once anchored to the surgical incision using the detachable anchor. Specifically, the expandable sleeve gets squeezed, when the trocar is maneuvered about the surgical incision, moving the inflating medium from one side to another. For example, when the trocar is tilted towards the left the inflating medium, within the expandable sleeve, move towards the right and vice-versa. This provides better maneuverability to the trocar about the surgical incision when used in the laparoscopic surgical procedure.

Further, at the end of the laparoscopic surgical procedure, the expandable sleeve can be deflated by removing the inflating medium from the expandable sleeve. In an example, the expandable sleeve can be deflated by drawing the inflating medium out of the expandable sleeve by the syringe. Alternatively, the elongated pipe can be decoupled from the connector for allowing the inflating medium to flow out of the expandable sleeve. Thereafter and once the guiding tool has been removed from the surgical incision, the detachable anchor can be detached from the guiding tool by simply rolling back the expandable sleeve towards its first end next to the hollow elongate member.

In yet another aspect, the present disclosure discloses a method for anchoring a guiding tool, such as a trocar, to a surgical incision made during a minimally invasive surgery. The method comprises arranging a detachable anchor according to the present disclosure over a shaft of the guiding tool, while the sleeve of the anchor is rolled up next to the hollow elongate member; unrolling the sleeve over the shaft of the guiding tool; inserting the guiding tool with the detachable anchor into the surgical incision; attaching an inflating medium outlet to the inflating medium inlet of the detachable anchor and inserting the inflating medium to the sleeve so that it inflates and thus retains the guiding tool in place in the surgical incision.

The method thus typically starts with arranging the detachable anchor according to the present disclosure over the shaft of the guiding tool, while the expandable sleeve of the detachable anchor is rolled up next to the hollow elongate member. Specifically, the guiding tool is inserted through the opening of the hollow elongate member such that the hollow elongate member rests on the shaft of the guiding tool.

At a further step, the method includes unrolling the sleeve over the shaft of the guiding tool. In this instance, the expandable sleeve due to its preferred elastic nature snugly secures the detachable anchor over the shaft of the guiding tool.

At a further step, the method includes inserting the guiding tool with the detachable anchor into a surgical incision. The surgical incision can be an incision made in a patient's body for performing a laparoscopic surgical procedure. Further, the incision utilizes a guiding tool for being inserted there-through to provide an access path for a surgical tool to be used in the laparoscopic surgical procedure.

At a further step, the method includes attaching the inflating medium outlet to the inflating medium inlet of the detachable anchor. Specifically, the detachable anchor is fluidically coupled to an inflating arrangement capable of delivering the inflating medium into the expandable sleeve. For example, an elongated pipe can be coupled to the connector of the detachable anchor, and the elongated pipe is further coupled to a syringe, which carries the inflating medium therein.

At a further step, the method includes inserting the inflating medium to the sleeve so that it inflates and thus retains the guiding tool in place in the surgical incision. For example, a plunger of the syringe can be pushed to inject the inflating medium out of the syringe. The inflating medium accordingly enters through the inlet of the hollow elongate member for being delivered to the expandable sleeve through the inlet of the expandable sleeve connected to the outlet of the hollow elongate member. The inflating medium could also be inserted directly to the inlet of the expandable sleeve, i.e. it is not obligatory that the inflating medium passes through the hollow elongate member. Therefore, the inner wall of the expandable sleeve comes into contact with the shaft of the guiding tool and the outer wall of the expandable sleeve comes into contact with body tissues along the surgical incision, for snugly securing the detachable anchor with the surgical incision. The guiding tool is accordingly snugly secured to the incision and provides an access path for a surgical tool to be used in the laparoscopic surgery procedure.

It will be appreciated that the above steps of the method as well as the embodiments of the device are only illustrative and other alternatives can also be provided where one or more steps are added, one or more steps are removed, or one or more steps are provided in a different sequence.

The embodiments of the present disclosure are thus directed towards a detachable anchor which can be used in a laparoscopic surgical procedure. Specifically, the detachable anchor can be used for anchoring a guiding tool, such as a trocar, to an incision made in the laparoscopic surgical procedure. In use, the detachable anchor can be easily arranged on the guiding tool, i.e., the guiding tool can be simply inserted through the detachable anchor and thereafter the expandable sleeve can be unrolled over the shaft of the guiding tool for snugly securing the detachable anchor on the guiding tool. The detachable anchor can snugly and firmly hold the guiding tool to the incision, when the expandable sleeve is inflated with the help of a medium using an inflating arrangement. This helps the surgeons to conveniently and confidently use surgical tools through the guiding tool during the laparoscopic surgical procedure because the detachable anchor limits the undesirable movements of the guiding tool with respect to the incision. For example, with a limited movement of the guiding tool with respect to the incision a surgeon can easily reach a target tissue, even when an upward or a downward force needs to be applied on the guiding tool for the manipulation of a surgical tool. Further, the detachable anchor hermetically seals the incision to avoid any leakage of the insufflation gas from around the guiding tools or through the incision during the laparoscopic surgical procedure. Moreover, the detachable anchor can be easily removed from the guiding tool at the end of the laparoscopic surgical procedure, allowing the guiding tool to be again used in conjunction with another detachable anchor in another laparoscopic surgical procedure. Therefore, the detachable anchor of the present disclosure provides a cost effective and/or economic solution for anchoring a guiding tool to an incision made in the laparoscopic surgical procedure.

### DETAILED DESCRIPTION OF THE DRAWINGS

The summary above, as well as the above and following detailed description of illustrative embodiments, is better understood when read in conjunction with the appended drawings. For the purpose of illustrating embodiments of the present disclosure, exemplary constructions of the disclosure are shown in the drawings. However, the present disclosure is not limited to specific a structural component and method for using the same as disclosed herein. Moreover, those in the art will understand that the drawings are not to scale. Wherever possible, like elements have been indicated by identical numbers.

Embodiments of the present disclosure will now be described, by way of example only, with reference to the following diagrams. In the accompanying drawings, a number relates to an item identified by a line linking the number to the item. When a number is accompanied by an associated arrow, the number is used to identify a general item at which the arrow is pointing.

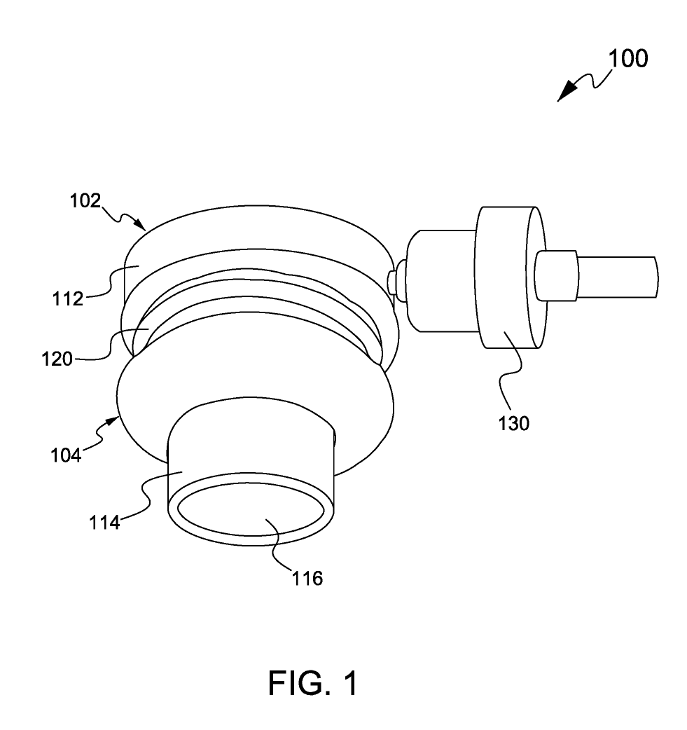

The present disclosure provides a detachable anchor for a guiding tool used in a minimally invasive surgery; and a method for anchoring the guiding tool using the detachable anchor to an incision made in the minimally invasive surgery. Referring now to the drawings, particularly by their reference numbers, FIG. 1 is an isometric view of a detachable anchor 100 for a guiding tool, in accordance with an embodiment of the present disclosure. The detachable anchor 100 includes a hollow elongate member 102 and an expandable sleeve 104 arranged on the hollow elongate member 102.

The hollow elongate member 102 includes a first portion 112 and a second portion 114 integrally and co-axially extending from the first portion 112. The hollow elongate member 102 also includes an opening 116, having a uniform diameter, passing through the first portion 112 and the second portion 114.

The expandable sleeve 104 is rolled up to fit over the second portion 114, next to the first portion 112, of the hollow elongate member 102. The expandable sleeve 104 is secured to the hollow elongate member 102 using a band 120. For example, an unrolled end (not numbered) of the expandable sleeve 104 close to the first portion 112 is secured to the second portion 114 using the band 120.

The detachable anchor 100 also includes a connector 130 connected to the first portion 112 of the hollow elongate member 102. The connector 130 is adapted to connect the hollow elongate member 102 to an inflating arrangement for inflating the expandable sleeve 104, which is further explained in detail in conjunction subsequent figures.

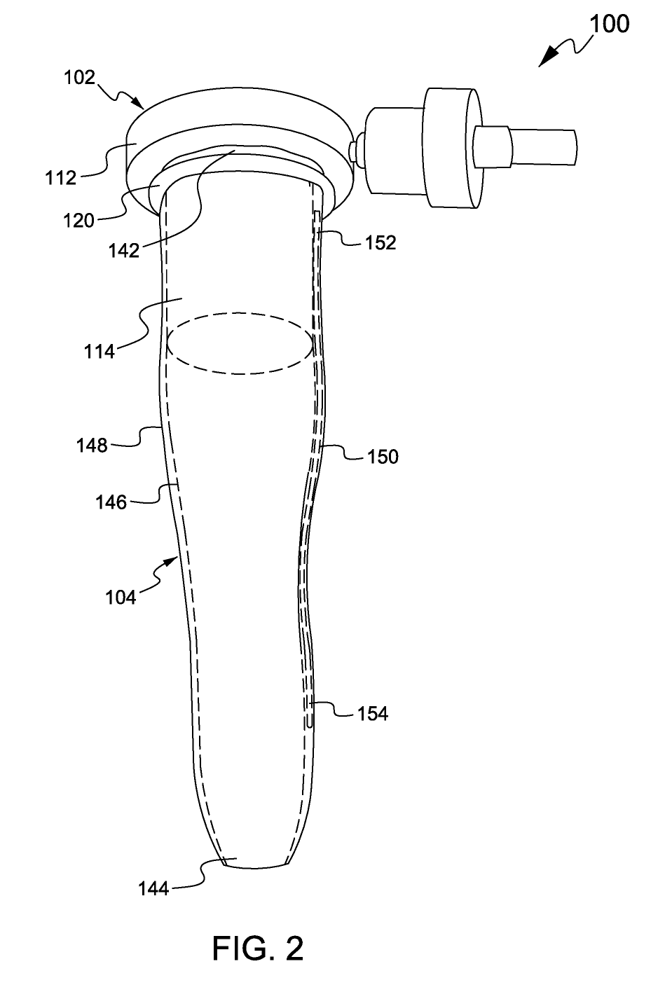

Referring now to FIG. 2, an isometric view of the detachable anchor 100 is shown with the expandable sleeve 104 in an unrolled state. The expandable sleeve 104 is a tubular structure having a first end 142 and a second end 144. The first end 142 of the expandable sleeve 104 is a proximal end with respect to the hollow elongate member 102 and the second end 144 of the expandable sleeve 104 is a distal end with respect to the hollow elongate member 102.

The expandable sleeve 104 is attached at its first end 142 to the hollow elongate member 102. For example, as explained above, the first end 142 of the expandable sleeve 104 can be secured to the second portion 114 next to the first portion 112 using the band 120.

Additionally, the expandable sleeve 104 is elastic in nature (made of an elastic biocompatible material); therefore the expandable sleeve 104 can snugly fit over the hollow elongate member 102. Further, the expandable sleeve 104 is a double wall structure including an inner wall 146 and an outer wall 148. The inner wall 146 and the outer wall 148 are attached to each other at the second end 144 of the expandable sleeve 104.

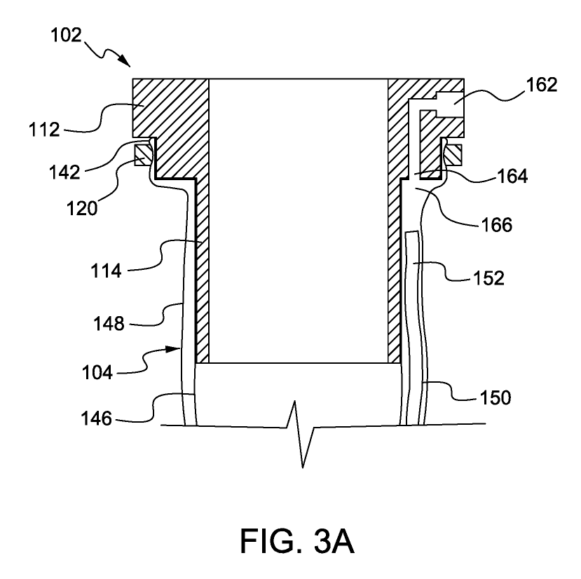

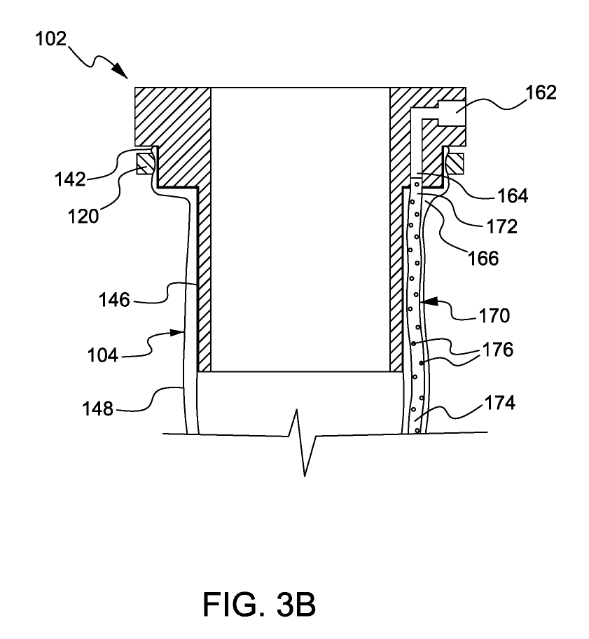

The detachable anchor 100 also includes a spacer 150 (shown with hidden lines), arranged between the inner wall 146 and the outer wall 148 of the expandable sleeve 104. The spacer 150 extends from its first end 152, which is the same end as the first end 142 of the sleeve 104, towards its second end 154 which is towards to second end 144 of the sleeve 104. Further, the spacer 150 extends partially along the length of the expandable sleeve 104, for example, the spacer 150 can extend at least 80% of the length of the expandable sleeve 104. The spacer 150 is configured to space apart the inner wall 146 and the outer wall 148 along its length thereof in the rolled and unrolled states of the expandable sleeve 104. The detachable anchor 100 can optionally include a tube instead of the spacer 150, which is further explained in detail in conjunction FIG. 3B.

Referring now to FIGS. 3A-3B, cross-sectional views of the hollow elongate member 102 of the detachable anchor are shown, in accordance with embodiments of the present disclosure. FIGS. 3A illustrates the first end 142 of the expandable sleeve 104 attached to the second portion 114 of the hollow elongate member 102 by the band 120. FIG. 3A also illustrates an inlet 162 and an outlet 164 for the hollow elongate member 102 arranged on the first portion 112. FIG. 3A further illustrates an inlet 166 for the expandable sleeve 104 at its first end 142. The outlet 164 for the hollow elongate member 102 is connected to the inlet 166 for the expandable sleeve 104. As shown, the outlet 164 is a small tubular projection coupled to the inlet 166 for the expandable sleeve 104.

Referring now to FIG. 3B, the detachable anchor is shown to optionally include a tube 170 arranged between the inner wall 146 and the outer wall 148 of the expandable sleeve 104. The tube 170 extends from its first end 172 towards its second end 174. The second end 174 of the tube 170 is actually proximate to the second end 144 (shown in FIG. 2) of the expandable sleeve 104, as the tube 170 is shown partially, labelling of the second end 174 should not be confusing. The tube 170 can also extend partially along the length of the expandable sleeve 104 similar to the spacer. Further, the tube 170 is connected to an inlet of inflating medium. For example, the first end 172 of the tube 170 is connected to the inlet 166 of the expandable sleeve 104 and to the outlet 164 for the hollow elongate member 102. The tube 170 includes a plurality of holes 176 arranged along a length of the tube 170.

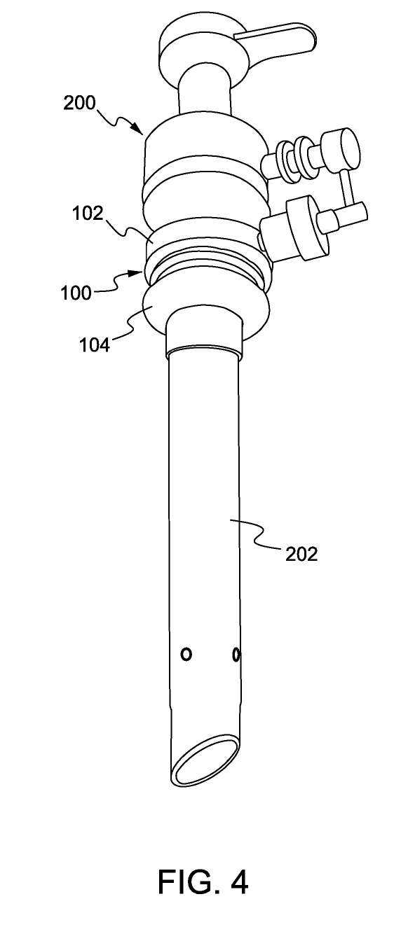

Referring now to FIG. 4, an isometric view of the detachable anchor 100 arranged on a guiding tool 200 (such as a trocar) is shown, in accordance with an embodiment of the present disclosure. The detachable anchor 100 is inserted through the guiding tool 200 to coaxially fit over a shaft 202 of the guiding tool. Specifically, the guiding tool 200 is inserted through the opening 116 (shown in FIG. 1) of the hollow elongate member 102 for allowing the detachable anchor 100 to rest on the shaft 202 of the guiding tool 200. Further, FIG. 4 illustrates the expandable sleeve 104 of the detachable anchor 100 in the rolled state.

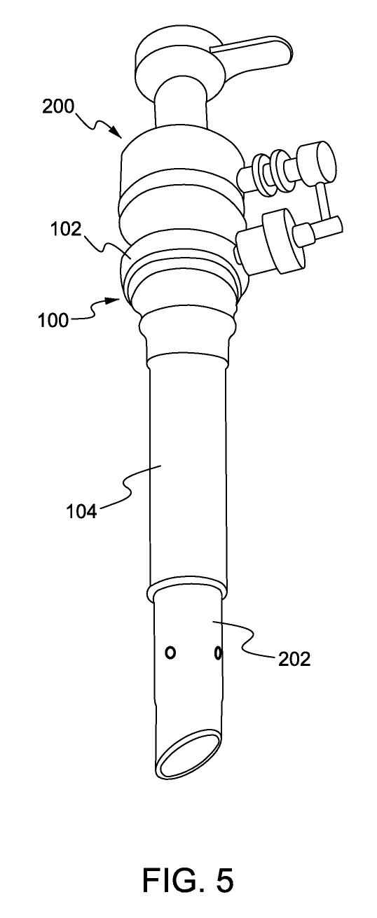

Referring now to FIG. 5, the detachable anchor 100 is shown to be arranged on the shaft 202 of the guiding tool 200 with the expandable sleeve 104 in the unrolled state, in accordance with an embodiment of the present disclosure. As shown, the expandable sleeve 104 is unrolled over the shaft 202 of the guiding tool 200 for substantially covering a length of the shaft 202. In this instance, the expandable sleeve 104 due to its elastic nature snugly fits over the shaft 202, and thereby snugly secures the detachable anchor 100 over the guiding tool 200.

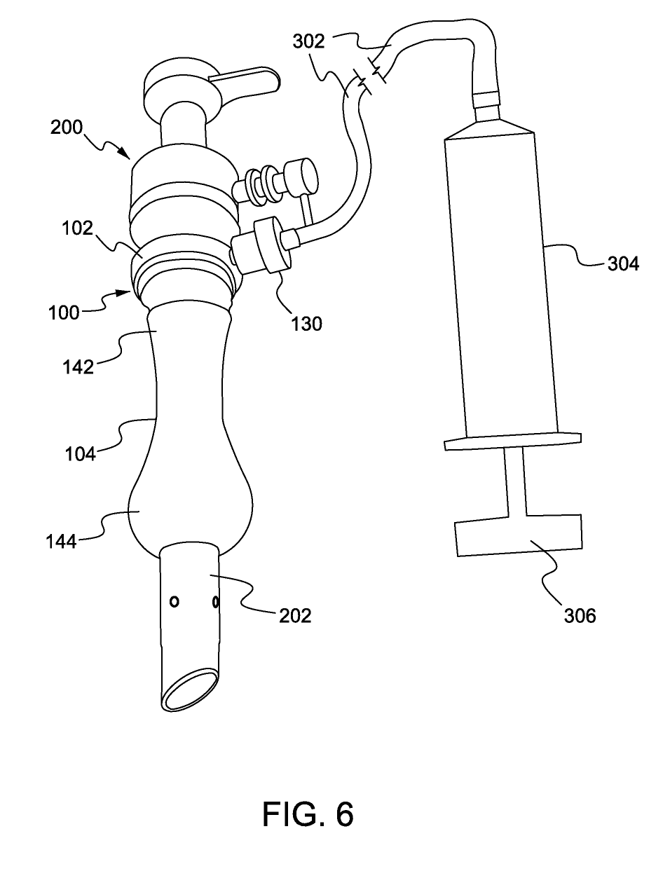

Referring now to FIG. 6, the detachable anchor 100 is shown to be arranged on the shaft 202 of the guiding tool 200 with the expandable sleeve 104 in an inflated state, in accordance with an embodiment of the present disclosure. Once the expandable sleeve 104 snugly fits over the shaft 202 of the guiding tool 200, the expandable sleeve 104 can be inflated with help of an inflating arrangement. In an example, the inflating arrangement includes an elongated pipe 302 adapted to be coupled to the connector 130 and a syringe 304 connected to the elongated pipe 302 and adapted to carry the inflating medium therein.

The inflating medium reaches the expandable sleeve 104 through a fluidic communication established between the syringe 304 and the expandable sleeve 104. Specifically, the inflating medium carried by the syringe 304 is delivered to the connector 130 with the help of the elongated pipe 302 when a plunger 306 of the syringe 304 is pushed to inject the inflating medium out of the syringe 304. Thereafter, the inflating medium enters through the inlet 162 (shown in FIGS. 3A-B) of the hollow elongate member 102 for being delivered to the expandable sleeve 104 through the inlet 166 (shown in FIGS. 3A-B) of the expandable sleeve 104. In case the detachable anchor 100 includes the tube 170 (shown in FIG. 3B) then the inflating medium is delivered into the expandable sleeve 104 through the holes 176 (shown in FIG. 3B) of the tube.

Once the inflating medium reaches the expandable sleeve 104, the expandable sleeve 104 attains the inflated state. The spacer 150 (shown in FIG. 2) or the tube 170 (shown in FIG. 3B), arranged between the inner wall and the outer wall of the expandable sleeve 104, can help the inflating medium to suitably flow along the length of the expandable sleeve 104 for inflation thereof. Specifically, the spacer or the tube provides small openings on either side thereof for allowing the inflating medium to easily flow along the length the expandable sleeve 104.

Further, in this embodiment, due to the varying thickness the expandable sleeve 104 along the length thereof, the expandable sleeve 104 attains a non-uniform inflation along the length thereof. As shown in FIG. 6, the second end 144 of the expandable sleeve 104 inflates more compared to the first end 142 of the expandable sleeve 104, when the inflating medium is delivered into the expandable sleeve 104. In an example, the expandable sleeve 104 is shown to attain a shape substantially similar to an hourglass shape.

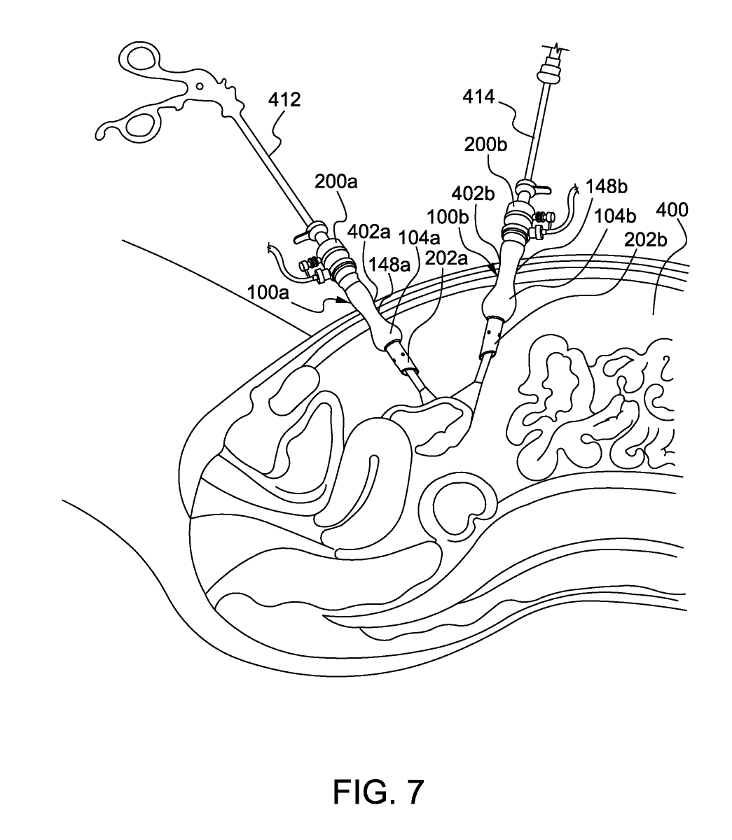

Referring now to FIG. 7, a sectional view of a female abdominal cavity 400 is shown during a laparoscopic surgical procedure with detachable anchors 100a and 100b anchoring guiding tools 200a and 200b (such as trocars) in surgical incisions 402a and 402b, in accordance with an embodiment of the present disclosure. The detachable anchors 100a and 100b hermetically anchor the guiding tools 200a and 200b in the incisions 402a and 402b. It is to be understood that the detachable anchors 100a and 100b shown herein are structurally and functionally similar to the detachable anchor 100, and therefore the detachable anchors 100a and 100b are explained herein later using the corresponding numerals of the detachable anchor 100.

The detachable anchors 100a and 100b are shown arranged on the guiding tools 200a and 200b, respectively. Further, the guiding tools 200a and 200b along with the detachable anchors 100a and 100b are shown inserted through the surgical incisions 402a and 402b, respectively. Moreover, the expandable sleeves 104a and 104b of the detachable anchors 100a and 100b are shown in the inflated state. In this instance, the inflated expandable sleeves 104a and 104b anchors the guiding tools 200a and 200b to the surgical incisions 402a and 402b, respectively. More specifically, the expandable sleeves 104a and 104b inflate in such a manner such that the outer walls of the expandable sleeves 104a and 104b snugly conform to the contours of the surgical incisions 402a and 402b, respectively, for snugly anchoring the guiding tools 200a and 200b to the surgical incisions 402a and 402b, respectively.

The expandable sleeves 104a and 104b are further shown to inflate more at their first ends as compared to their second ends (this is due to the varying thickness of the expandable sleeves 104a and 104b along their lengths). The guiding tools 200a and 200b (anchored to the surgical incisions 402a and 402b with the detachable anchors 100a and 100b) provide access paths for the surgical tools to be used in the laparoscopic surgical procedure. The surgical tools, such as a gasper 412 and a light emitting device 414, can be inserted through the guiding tools 200a and 200b to reach the target tissues. As shown in FIG. 7, the surgical tools are used for performing a gynaecological laparoscopic procedure using the access paths provided by the guiding tools 200a and 200b.

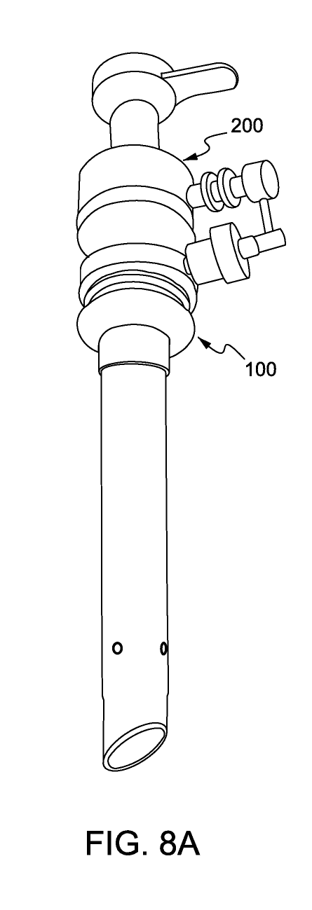

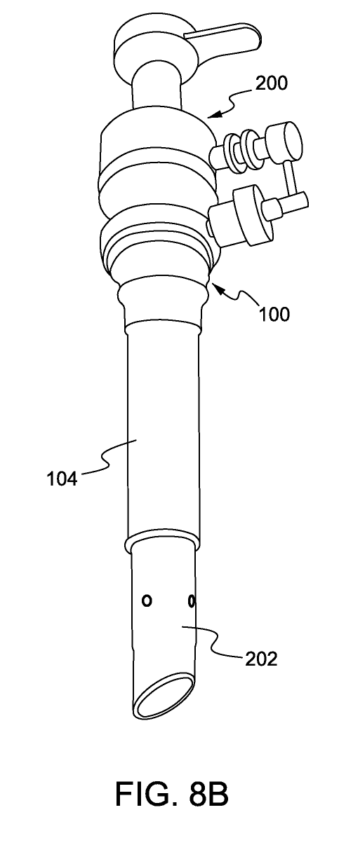

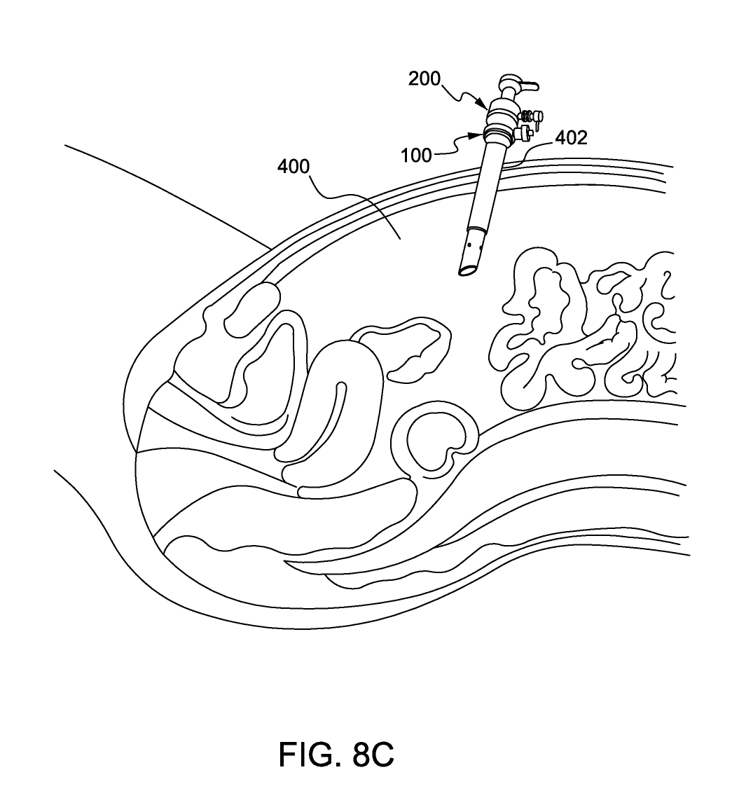

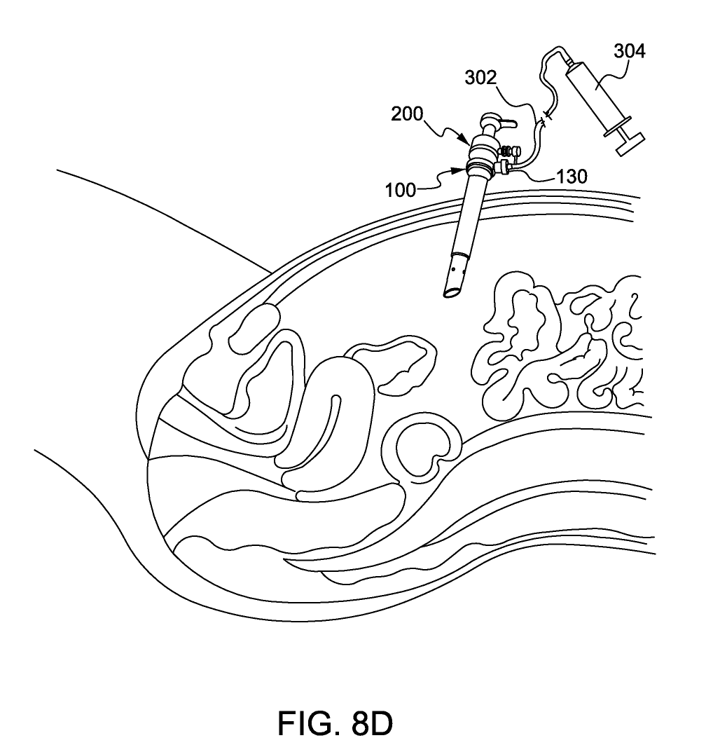

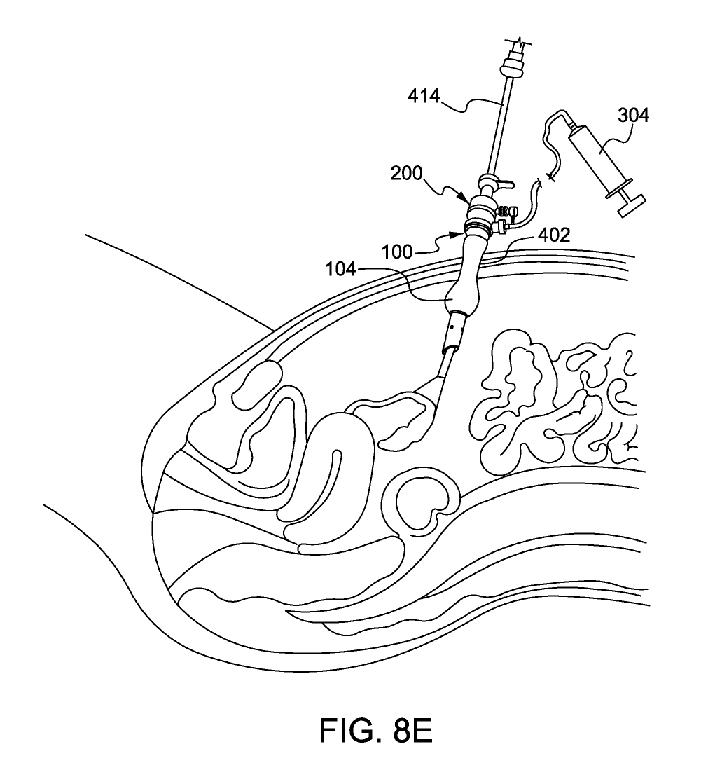

Referring now to FIGS. 8A-E, steps of an exemplary method for anchoring a guiding tool (such as a trocar) to a surgical incision is shown, in accordance with an embodiment of the present disclosure. More specifically, the FIGS. 8A-E illustrate exemplary steps to be followed by the detachable anchor 100 for anchoring the guiding tool 200 to an incision of a laparoscopic surgical procedure. In FIG. 8A, the detachable anchor 100 is arranged on the guiding tool 200. In FIG. 8B, the expandable sleeve 104 of the detachable anchor 100 is unrolled over the shaft 202 of the guiding tool 200 for snugly securing the detachable anchor 100 over the guiding tool 200. In FIG. 8C, the guiding tool 200 with the detachable anchor 100 secured thereto is inserted through the surgical incision 402. Specifically, the guiding tool 200 is inserted through a surgical incision 402 to a desired depth into the abdominal cavity 400. In FIG. 8D, an inflating medium outlet, such as an elongated pipe 302, is attached to an inflating medium inlet, such as the connector 130, of the detachable anchor 100. The elongated pipe 302 is connected to the syringe 304, which carries the inflating medium therein. In FIG. 8E the inflating medium is inserted into the expandable sleeve 104 so that the expandable sleeve 104 inflates and thus retains the guiding tool 200 in place in the surgical incision 402. For example, the syringe 304 may be operated to inject the inflating medium stored therein for being delivered to the expandable sleeve 104. The guiding tool 200 accordingly provides an access path for the surgical tool 414 to be used in the laparoscopic surgical procedure.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

All directional references (e.g., upper, lower, upward, downward, left, right, leftward, rightward, top, bottom, above, below, vertical, horizontal) are only used for identification purposes to aid the reader's understanding of the present disclosure, and may not create limitations, particularly as to the position, orientation, or use of the detachable anchor and/or methods disclosed herein. Joinder references (e.g., attached, affixed, secured, coupled, connected, hinged, and the like) are to be construed broadly and may include intermediate members between a connection of segments and/or suggest relative movement between segments. As such, joinder references do not necessarily infer that two segments are directly connected and in fixed relation to each other.

Additionally, all numerical terms, such as, but not limited to, “first”, “second”, “third”, or any other ordinary and/or numerical terms, should also be taken only as identifiers, to assist the reader's understanding of the various embodiments, variations and/or modifications of the present disclosure, and may not create any limitations, particularly as to the order, or preference, of any embodiment, variation and/or modification relative to, or over, another embodiment, variation and/or modification.

While certain exemplary embodiments have been described and shown in the accompanying drawings, it is to be understood that such embodiments are merely illustrative of and not restrictive on the broad present disclosure, and that this present disclosure not be limited to the specific constructions and arrangements shown and described, since various other modifications and/or adaptations may occur to those of skill in the art. It is to be understood that individual features shown or described for one embodiment may be combined with individual features shown or described for another embodiment. It is to be understood some features are shown or described to illustrate the use of the present disclosure in the context of functional segments and such features may be omitted within the scope of the present disclosure and without departing from the spirit of the present disclosure as defined in the appended claims.

1. A detachable anchor for a guiding tool of a minimally invasive surgery, comprising a hollow elongate member arranged to coaxially fit over a shaft of the guiding tool, and
an expandable sleeve
having a first end and a second end,
having at least partially double wall structure consisting of an inner wall and an outer wall, wherein the walls are attached to each other at least at the second end of the sleeve,
wherein the expandable sleeve is attached at its first end to the hollow elongate member, and
includes an inlet for inflating medium.

2. A detachable anchor according to claim 1, wherein the hollow elongate member comprises a first portion, a second portion integrally extending from the first portion and an opening extending through the first portion and the second portion, wherein the first portion and the second portion are co-axial.

3. A detachable anchor according to claim 1 wherein the hollow elongate member comprises an inlet and an outlet for inflating medium, the outlet being connected to the inlet for inflating medium in the sleeve.

4. A detachable anchor according to claim 3, wherein the inlet of the hollow elongate member is arranged to be coupled to a connector.

5. A detachable anchor according to claim 1, wherein the thickness of at least one of the inner and outer walls of the sleeve varies along its length.

6. A detachable anchor according to claim 5, wherein the thickness of at least one of the inner and outer walls of the sleeve is smaller at its second end than at its first end.

7. A detachable anchor according to claim 5 wherein the thickness of only the outer wall varies along its length.

8. A detachable anchor according to claim 5, wherein the expandable sleeve is arranged to inflate non-uniformly when inflated using an inflating medium.

9. A detachable anchor according to claim 1, wherein the sleeve is arranged to be rolled up towards the first end.

10. A detachable anchor according to claim 1, wherein a band is arranged to secure the first end of the expandable sleeve to the hollow elongate member.

11. A detachable anchor according to claim 1, wherein a spacer is arranged between the inner and outer walls of the sleeve, extending from the first end towards the second end.

12. A detachable anchor according to claim 11, wherein the spacer is configured to distribute the inflating medium evenly along the length of the expandable sleeve.

13. A detachable anchor according to claim 11, wherein the spacer extends on at least 80% of the length of the sleeve.

14. A detachable anchor according to claim 1, further comprising a tube for conducting the inflating medium is arranged between the inner and outer walls of the sleeve, extending from the first end towards the second end, the tube being connected to the inlet of inflating medium.

15. A detachable anchor according to claim 1, wherein the sleeve comprises elastic polymeric material or elastic composite polymeric material.

16. A detachable anchor according to claim 1, wherein the hollow elongate member comprises metal, glass, ceramic, plastic, fiber-reinforced plastic or any combination thereof.

17. A detachable anchor according to claim 11, wherein the spacer comprises flexible polymeric material or flexible composite polymeric material.

18. A detachable anchor according to claim 1, wherein the expandable sleeve is arranged to be unrolled over a shaft of the guiding tool for snugly securing the detachable anchor to the guiding tool.

19. A detachable anchor according to claim 18, wherein the sleeve is arranged to be rolled up towards the first end next to the hollow elongate member for detaching the detachable anchor from the guiding tool.

20. A method for arranging a detachable anchor comprising:
providing a detachable anchor having:
a hollow elongate member arranged to coaxially fit over a shaft of a guiding tool, and an expandable sleeve including a first end, a second end and at least partially double wall structure with an inner wall and an outer wall, wherein the walls are attached to each other at least at the second end of the sleeve, wherein the expandable sleeve is attached at its first end to the hollow elongate member, and has an inlet for inflating medium;
arranging the detachable anchor over a shaft of a guiding tool, while the sleeve of the detachable anchor is rolled up next to the hollow elongate member; and
unrolling the sleeve over the shaft of the guiding tool.
